(19) United States
(12) Patent Application Publication
(10) Pub. No.: US 2006/0257932 A1
(43) Pub. Date: Nov. 16, 2006

(54) Treatment of Crohn's disease

(76) Inventors: Yaron Ilan, Givat Massua (IL); Maya Margalit, Abu Gosh (IL)

Correspondence Address: ENZO BIOCHEM, INC., 527 MADISON AVENUE (9TH FLOOR), NEW YORK, NY, 10022, US

(21) Appl. No.: 11/378,158

(22) Filed: Mar. 16, 2006

Related U.S. Application Data
(63) Continuation-in-part of application No. 09/356,294, filed on Jul. 16, 1999.
Application No. 09/356,294 is a division of application No. 08/808,629, filed on Feb. 28, 1997, now abandoned.

(51) Int. Cl. G01N 33/53; G01N 33/00

(52) U.S. Cl. 435/7.1; 436/86

(57) ABSTRACT

The oral administration of a drug results in the safe and effective treatment of individuals with moderate to severe Crohn's disease. Additionally, the identification and measurement of the levels of certain markers may be used to create an immune profile for the prediction of clinical responses to the drug. These measurements may also be developed into surrogate markers for the response to the treatment of the disease.

### REFERENCE TO RELATED PATENT APPLICATIONS

This application is a continuation-in-part of U.S. patent application Ser. No. 09/356,294, filed on Jul. 16, 1999, entitled “Novel Selective Immune Regulation (SIDR) Mediated Transplantation Processes, Processes for Preventing or Treating Diseases in a Subject, and Compositions of Matter Comprising Trained or Programmed Cells, Tissues or Organs Useful for SIDR Establishment”, which is a divisional of U.S. patent application Ser. No. 08/808,629, filed on Feb. 28, 1997, entitled “Novel Processes Implementing Selective Immune Down Regulation (SIDR)”. The content of the aforementioned patent applications are hereby incorporated by reference, in their entirety.

### FIELD OF THE INVENTION

This invention relates to the use of a drug in the treatment of Crohn's disease, and the identification of certain markers that may be used to create an immune profile for the prediction of clinical responses to the drug.

All patents, patent applications, patent publications, scientific articles, and the like, are hereby incorporated by reference in their entirety in order to describe more fully the state of the art to which the present invention pertains.

### BACKGROUND OF THE INVENTION

Crohn's disease is an idiopathic, immune-mediated disorder characterized by a chronic relapsing-remitting course (Podolsky, D. K., N Engl J Med 347, 417-429 (2002)). Although the pathogenesis of Crohn's disease has not been adequately clarified, current understanding is that the transmural inflammation, the primary presentation of Crohn's disease, is the result of a cascade of events and processes initiated by one or more antigens as yet unspecified. This pathogenic immune response may be elicited by and act on an unidentified epitope(s) or it may simply be an inappropriate response to an otherwise innocuous epitope(s) that acts on a secondary target (Sands, B. E., Gastroenterology 118, 68-82 (2000)). While several new strategies of medication for the treatment for Crohn's disease have been developed recently (Egan L. J. & Sandborn, W. J., Gastroenterology 126, 1574-1581 (2004)), all currently available strategies of medication are still based on the systemic suppression of certain aspects of the immune system which can and do lead to significant drug-related adverse responses. Thus, there continues to be a need for a therapeutic strategy that is more specific and less global in its effect on the immune system.

The oral administration of specific antigens (oral immune regulation) can lead to the alteration of the specific immune response towards antigens (including disease-associated antigens), while not contributing to any significant drug related adverse responses (Weiner, H. L., Immunol Today 18, 335-343 (1997); Strober, S. & Mowat, A. M., Immunol Today 4, 173-81 (1998)). This procedure has been shown to be effective in preventing or managing immune-mediated disorders in various animal models (Miller A., Lider, O., Roberts, A. B., Sporn M. & Weiner, H. L., Proc. Natl. Acad. Sci. (USA) 89, 421-425 (1992); Von Herrath, M. G., Dyrberg, T. & Oldstone, M. B. A., J Clin Invest 98, 1324-1331 (1996); Ilan Y. et al., J Clin. Invest 99, 1098-1106 (1997)).

In one system, experimental colitis was stimulated in rodent populations treated with the chemical 2,4,6-trinitrobenzene sulfonic acid (TNBS). This autoimmune response resembles Crohn's disease in human patients (Strober W., et al., Immunol Today 18, 61-64 (1997)). Using this system, it was shown that the oral administration of low doses of colon-extracted antigens to experimental colitis mice significantly decreased the inflammatory response (Neurath, M., Kelasall, B. L., Presky, D. H., Waegell, W. & Strober, W., J Exp Med 183, 2605-2616 (1996); Ilan, Y. et al., Am J of Gastroenterology 95, 966-973 (2000)). Furthermore, oral administration of autologous (from syngeneic mice) colon-extracted antigens proved to be more effective than colon-extracted antigens from another species of animal and even from other strains of mice (Gotsman, I., Shlomai, A., Alper, R., Rabbani, E., Engelhardt, D. & Ilan, Y., J Pharmacology and Exp Therapeutics 297, 926-932 (2001); Shlomai, A. et al., J of Pathology 195, 498-507 (2001)). This data supports the conclusion that autologous colon-extracted antigens could be an effective therapeutic for managing Crohn's disease. In line with this, colon-extracted antigens manufactured from autologous mucosal extract are the preferred source of the drug in the management of Crohn's disease by oral immune regulation.

### SUMMARY OF THE INVENTION

In summary, the results of the present study suggest that the oral administration of Alequel™, a form of personalized medicine, is a safe and effective method for the treatment of individuals with moderate to severe Crohn's disease in this subject population. In addition, measurements of the levels of several immune markers demonstrated that an immune profile may exist that may predict the likelihood of a positive clinical response to the study drug. In addition, certain of these cellular measurements may be developed into surrogate markers for the response to the treatment of the disease.

### BRIEF DESCRIPTION OF THE FIGURES

FIG. 1:

Average CDAI during the course of the study.

A. CDAI in the drug-treated group. The solid line indicates the average Crohn's Disease Activity Index (CDAI) of those subjects who reached clinical remission (DR); the broken line indicates those who did not reach clinical remission (DNR).

B. CDAI in the placebo-treated group. The solid line indicates those subjects in the placebo treated group who reached clinical remission (PR); the broken line indicates those who did not reach clinical remission (PNR).

FIG. 2:

Effect of study drug on the percentage difference in IBDQ score between the initiation of treatment (week 0) and termination of treatment (week 15): DR indicates those subjects in the drug treated group who reached clinical remission and DNR indicates those who did not reach clinical remission. PR indicates those subjects in the placebo group who reached clinical remission and PNR indicates those who did not reach clinical remission.

FIG. 3:

Subject specific antigen-directed IFNγ ELISPOT: IFNγ spot forming cells (SFC) were identified by a modified subject-specific antigen-directed ELISPOT assay using the study drug specific to each subject. The results shown here are those obtained when the study drug was used at a concentration of 10 μg/ml. The drug-treated group is indicated by a circle, with the solid line indicating DR and the broken line indicating DNR; the placebo treated group is indicated by a triangle, with the solid line indicating PR and the broken line indicating PNR.

FIG. 4:

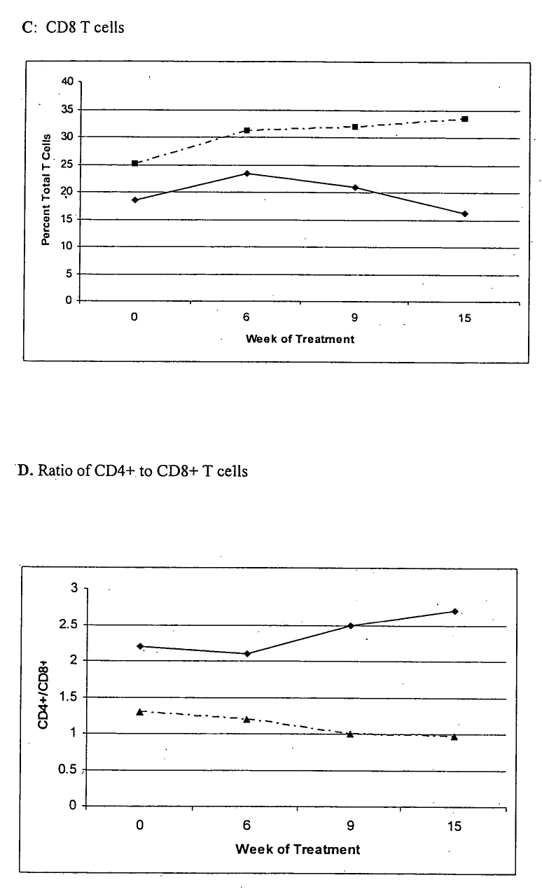

Effect of study drug on peripheral blood T cell subpopulations in the drug treated subjects: A. NKT lymphocytes; B. CD4 T cells; C. CD8 T cells; D. CD4/CD8 ratio proportion of PBMC. Solid lines indicate those subjects who reached clinical remission (DR); open lines indicate those subjects who did not reach clinical remission (DNR).

### DETAILED DESCRIPTION OF THE INVENTION

In a recently conducted phase I clinical trial the safety and efficacy of the oral administration of subject-derived intestinal antigen(s) colon-extracted antigens (CEA) for the treatment of Crohn's disease in 10 subjects was examined (Israeli, E. et al., World Journal of Gastroenterology (in the press)). The study drug was individually prepared from mucosal tissue obtained through colon biopsies from each of 10 subjects. At the close of this phase I trial the oral administration of the autologous study drug (Alequel™) was found to be safe within the limits of the number of subjects tested. Furthermore, during the course of the trial, remission defined by a decrease in the Crohn's Disease Activity Index (CDAI) score to 150 or lower was achieved in 7 out of 10 subjects.

The phase II study was intended to further evaluate the safety and efficacy of the oral administration of this personalized study drug to subjects with Crohn's disease in a randomized, double blind, placebo-controlled format. In addition, several markers were evaluated that could be used to describe an immune profile for individual persons that would predict which of these individuals would have the capacity to produce an efficacious response to the administration of the study drug.

### EXAMPLES

Materials and Methods

Patient population: A randomized double blind, placebo-controlled, one-center trial was conducted in subjects with moderate to severe Crohn's disease.

Inclusion criteria: Participants (men and women ≧18 years) were evaluated for eligibility after executing a written informed consent. The diagnosis of Crohn's disease with clinical evidence of active (symptomatic) disease was based on clinical history, blood tests and/or histology, x-ray, or endoscopy. Subjects were required to have a CDAI score between 220 and 400 as a condition for enrollment. Subjects who were receiving oral steroid therapy at the time of enrollment were required to be on a dose regimen equivalent to <25 mg of prednisone per day.

Exclusion criteria: Subjects who had undergone bowel surgery within the last 3 months; who had had a prior colostomy, ileostomy, or colectomy with ileorectal anastamosis; those subjects whose symptoms were believed to be due to the presence of fibrotic strictures; or those who were likely to require emergency surgery for persistent intestinal obstruction, bowel perforation, uncontrolled bleeding, abdominal abscess, infection, or toxic megacolon were ineligible for entry into the study. In addition, subjects with an infectious or neoplastic disease were ineligible. Subjects on a dose regimen of oral steroid therapy equivalent to greater than 25 mg of prednisone per day, subjects on a regimen of immunosuppressive drugs such as azathioprine or 6-mercaptopurine, or subjects receiving an elemental diet or parenteral nutrition were ineligible. In addition, subjects who had been treated with methotrexate, cyclosporine or anti-TNFα or who had participated in another clinical trial within 3 months prior to enrollment were ineligible.

Study drug preparation and administration: Subjects who fulfilled the inclusion/exclusion criteria for participation in the study were scheduled for a colonoscopy during which colon biopsies were removed for preparation of the colon-specific antigen-containing extract (study drug). The study drug was prepared individually for each subject in 5 ml doses in phosphate buffered saline and stored at −20° C. The administered dose ranged from 3 to 14 μg of protein. The placebo contained 5 ml of saline. Each subject followed a regimen of 3 doses of autologous study drug per week for 15 weeks, for a total of 45 doses.

Randomization: Subjects were randomized by a computer-generated randomization program to receive either the study drug or the placebo. All subjects and investigators were blinded for treatment allocation. The blinding code was kept confidential by an independent statistician.

Clinical and laboratory follow-up: Study subjects were monitored by a variety of clinical, laboratory and quality of life parameters during the treatment period (weeks 0-15), and follow-up period after treatment (weeks 16-27). The safety and tolerability of the oral administration of the study drug was assessed by evaluating the clinical parameters detailed by the subject in his/her diary entries. During the treatment period, a designated study physician was accountable for interim history, physical examination and adverse event assessment every three weeks. Blood was drawn at each visit for complete blood counts (CBC), sedimentation rate (ESR) and standard chemistries (SMA). During the follow-up period, medical history, physical examination and laboratory tests were performed every 4 weeks. The evaluation of the effect of the study drug on the clinical status of the subjects was assessed by following the CDAI score obtained for the week prior to the clinic visit. The Inflammatory Bowel Disease Questionnaire (IBDQ) filled out by each subject, was evaluated at baseline and at the end of the treatment period (15 weeks). The primary endpoint was complete clinical remission, defined as a decrease in a CDAI score to 150 or less. Secondary efficacy criteria included decreases in CDAI, and analysis of the IBDQ, a perceived improvement in quality of life.

As a means for identifying a possible surrogate marker(s) to assess the clinical effect of the study drug, specific antigen-directed IFN gamma production (by ELISPOT assay), T cell proliferation and FACS analysis of peripheral blood T cell populations were performed on specimens obtained at weeks 0, 9 and 15.

Flow cytometry analysis for determination of the effect of oral immune regulation on CD4, CD8, NKT lymphocytes in peripheral blood: Blood samples were collected throughout the study period. Immediately after lymphocyte isolation, duplicates of 2-5×10⁴ cells/500 μl PBS were deposited into Falcon 2052 tubes incubated with 4 ml of 1% BSA for 10 minutes, and centrifuged 1400 rpm for 5 minutes. Cells were resuspended in 10 82 l FBS with either 1:20 FITC-anti human CD3, CD4, CD8, CD16 or CD56 antibodies (Pharmingen, and R&D, USA), and mixed every 10 minutes for 30 minutes. After the cells were washed twice with 1% BSA, 0.5 ml of 1% paraformaldehyde was added, and the cells were kept at 4° C. until reading. For the control group, only 5 μl of 1% BSA was added. Cell phenotyping was performed by a FACSTAR plus, (Becton Dickinson). Only live cells were counted, and background fluorescence from non-antibody-treated lymphocytes was subtracted.

Subject specific antigen-directed IFNγ ELISPOT assays: IFNγ spot forming cells (SFC) were identified using a modified subject-specific antigen-directed ELISPOT assay (Mabtech, Nacka, Sweden) (Gotsman, I. et al. Induction of oral tolerance towards hepatitis B envelope antigens in a murine model. Antiviral Research 48, 17-26 (2000)). Filtration plates (96 well), coated with high protein binding hydrophobic PVDF membrane (polyvinylidene disulfide), were used (Millipore Corp., Bedford, Mass., USA). Plates were coated with 1-D1K anti-IFNγ coating antibody (15 mg/ml, Mabtech, Nacka, Sweden) for 24 hours at 4° C. Peripheral blood mononuclear cells (PBMC) were isolated by Ficoll gradient separation of 20 ml blood samples, collected in acid citrate dextrose tubes, and processed within 1 hour. PBMC were washed twice in RPMI 1640 with 10% fetal bovine serum. Cells were cultured in 96 well plates (1×10⁵ cells/well) with RPMI 1640 and 10% FBS. Triplicate samples were prepared with 2 doses each of the study drug from each subject (5 and 10 82 g/ml) or phytohemagglutinin (PHA, 2.5 μg/ml) without antigen. Plates were incubated for 48 hours at 37° C. with 5% CO2. The plates were washed and 100 μl biotinylated antibody (7-B6-1-biotin, Mabtech, Nacka, Sweden) at a concentration of 1 μg/ml in filtered PBS with 0.5% FBS was added. Plates were incubated for 3 hours at room temperature. Following washing, 100 μl of streptavidin-alkaline phosphatase was added, and plates were incubated for 90 minutes at room temperature. The plates were washed and substrate (BCIP/NBT) from BioRad, Richmond, Calif.) was added for 30 minutes until red-purple spots appeared. Using a dissection microscope, dark spots, reflecting IFNγ-secreting clones were counted independently by two investigators. Results are expressed as means of triplicate IFNγ-secreting cells per 10⁵ PBMC, after subtraction of the mean spots from wells without study drug.

Statistical Analysis:

Sample size and power. Thirty one subjects were enrolled, randomized and treated according to the protocol. The study was not powered to detect rarely occurring treatment-associated adverse events.

Summary statistics by time point of all clinical and laboratory variables were calculated, and statistical significance of changes from baseline was assessed by student t-tests.

Results

Population: 35 potential subjects were screened; 31 of these subjects met all inclusion and exclusion criteria and were randomized, 15 subjects received the placebo and 16 subjects received the study drug. One subject withdrew consent after enrollment and was not treated. Four subjects (3 study drug and 1 placebo) experienced an exacerbation of their disease within the initial weeks of the study and their participation was subsequently terminated. One additional subject was unable to be evaluated since the material obtained from the biopsy did not produce a sufficient quantity of protein.

Subject characteristics: The data from the two groups of evaluable subjects were analyzed; one group compromising 12 subjects received the study drug, and the second group comprising 14 subjects received the placebo (see Table 1). The drug group included 3 males and 9 females. The mean age was 30.6 years (range: 20-47). The placebo group included 7 males and 7 females; the mean age was 33.0 years (range: 18-56). The mean duration of disease was 8.7 years (range: 3-17) in the study drug group and 8.6 years (range: 2-23) in the placebo group. The disease site was the small intestine, large intestine or both in 4, 2, and 6 subjects in the study drug group and 5, 1, and 8 subjects in the placebo group, respectively. Three subjects in the drug group and 4 subjects in the placebo group were on a regimen of corticosteroids (less than an equivalent of 25 mg prednisone) at the initiation of treatment.

CDAI score: The average CDAI of all enrolled subjects at the initial treatment visit was 304 with a maximum of 394 and a minimum of 230. The average initial CDAI of those subjects randomized to receive placebo was 301 with a maximum of 391 and a minimum of 231, while the initial CDAI of those subjects who would receive the study drug was 311 with a maximum of 394 and a minimum of 236.

Clinical remission (Table 2): Clinical remission, defined as a decrease to a CDAI of 150 or lower in 2 consecutive visits during the study period, was used as the primary measure of efficacy. 7 of the 12 evaluable subjects (58%) who received the study drug achieved a CDAI of 150 or lower, while 4 of the 14 evaluable subjects (29%) in the placebo group achieved remission.

Clinical response (Table 2): Clinical response was defined as a decrease of the CDAI score ≧100 points from baseline in 2 consecutive visits during the study period. 67% of the drug-treated subjects (8 out of 12) achieved this point, compared to 43% (6 out of 14) of the placebo group.

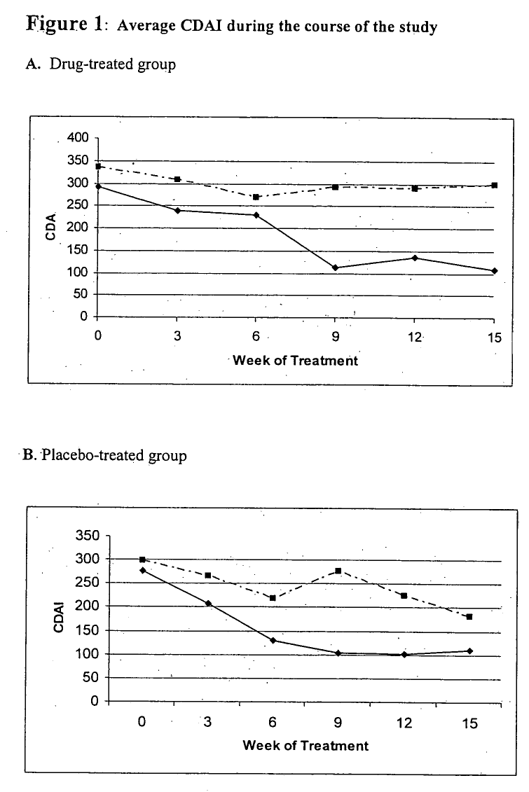

One feature of these data is the appearance of an inflection point between 6 and 9 weeks of medication in the drug treatment group of subjects who achieved clinical remission (DR) when the CDAI score is plotted against time (FIG. 1A,B). Such an inflection point was not seen in the drug treatment group which did not achieve clinical remission (DNR) or in the placebo groups.

IBDQ: (Table 2): Quality of life, as a supplementary indication of efficacy, was assessed by the IBDQ, an evaluation of health perception and function, completed by each subject before and at the end of treatment. The IBDQ score improved an average of 43% in all subjects who received the study drug and 61% in the DR group of subjects. The IBDQ score improved 2% in all subjects who received placebo and 10% in those who received the placebo and achieved clinical remission during the course of the trial (PR). When the extent of improvement of the DR group of subjects was compared with the PR group, a significant difference was observed (p=0.008).

Immune profile of the drug group subjects who achieved clinical remission: Several clinical parameters were monitored during the course of the trial. A novel difference was found among DR subjects and the DNR subjects. The differences suggest that the subjects who became part of the DR group and the subjects who became part of the DNR group may have had different physiological profiles at the initiation of treatment.

CRP level: The average level of circulating C-reactive protein (CRP) before the initiation of treatment was lower in the DR subjects (1.5 mg/L) than in the DNR cohort (6.1 mg/L; p=0.02). The cohort of subjects in the placebo group who reached remission during the course of the study (PR) as well as subjects in the placebo group who did not reach remission (PNR) both had average CRP levels of 3 mg/L.

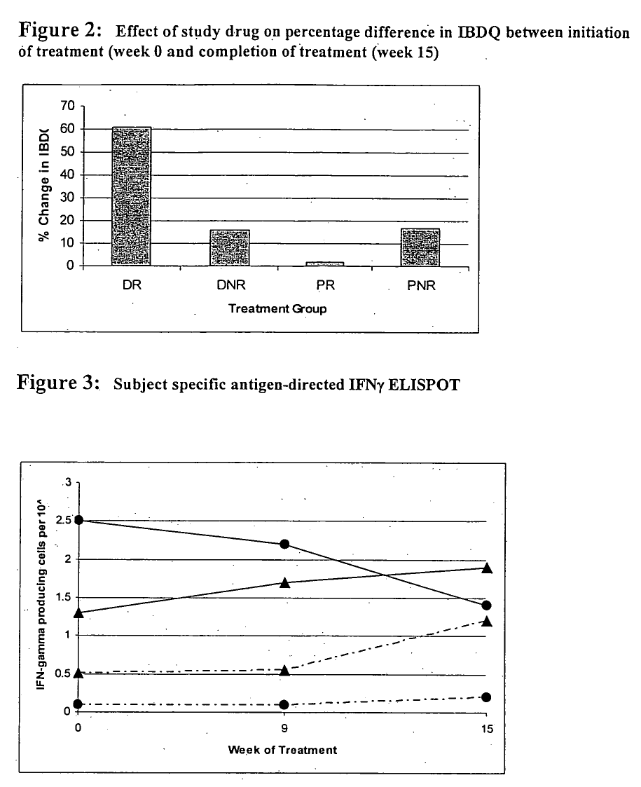

Subject specific antigen-directed IFNγ ELISPOT: The results of the ELISPOT assay for IFNγ-producing colonies (SFC) from the drug and placebo treated subjects at various times during the trial is shown in FIG. 2. These results suggest that the DR group had as many, or more such colonies before initiation of treatment compared with the DNR group. They further suggest that the number of such colonies declined throughout the treatment period while the number of IFNγ-producing colonies from the DNR group as well as both placebo groups (PR and PNR) increased.

Peripheral Blood Lymphocyte Subpopulations:

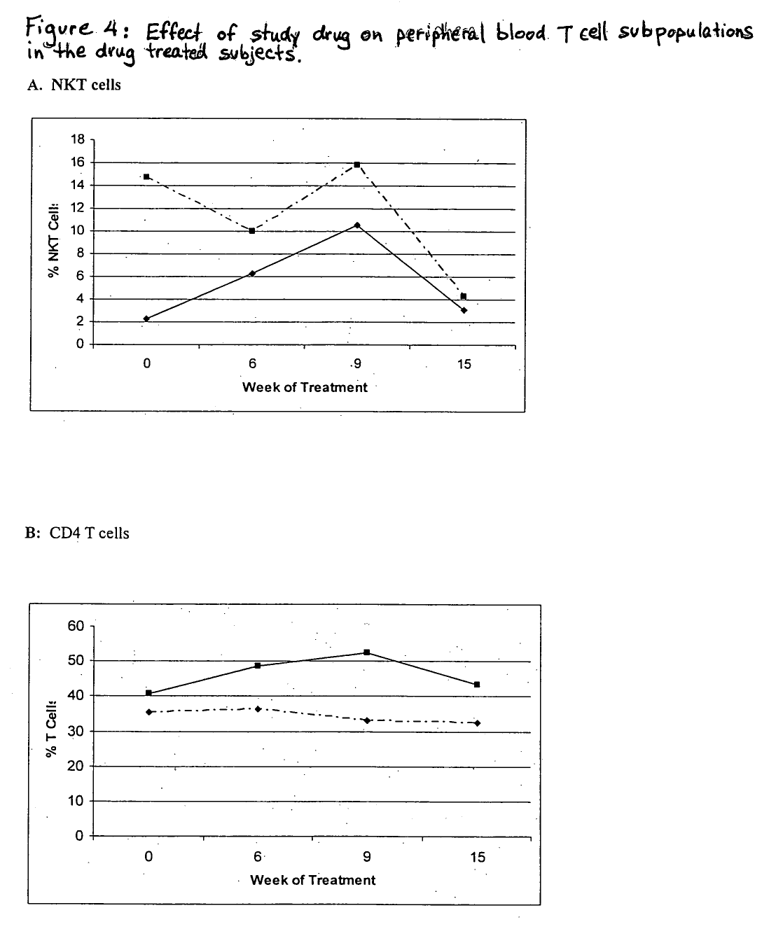

NKT lymphocytes: The results of periodic measurement of circulating NKT cells are reported in FIG. 3A. Before initiation of treatment, the proportion of T cells that were NKT cells in the DR group (2.3%) was significantly lower than that of the DNR group (14.8%; p=0.0051). These data support the conclusion that there is a difference in the immunological profile of each these groups of subjects at the initiation of treatment. The proportion of peripheral NKT cells in the DR group increased to 10.5% between weeks 0 and 9. No significant differences were observed between the proportion of NKT cells in the two placebo groups (PR and PNR). For the PR group the percentage of NKT cells before the initiation of treatment was 5% while at 9 weeks it was 4.3%. For the PNR group, the percentage of NKT cells at initiation of treatment was 5.3% and at 9 weeks it was 4.6%. Interestingly, the DNR group showed a significant (p=0.04) decrease in the percentage of NKT cells from 14.8% at the initiation of treatment to 4.3% after 15 weeks of treatment. Thus, while there may be a significant difference between the DR group and the DNR group before the initiation of treatment, this failure of response of the DNR group may be of a cyclical nature rather than one of a stable nature.

CD4+ T cells: As can be seen in FIG. 3B there is a difference between the percentage of CD4+ T cells at the initiation of treatment between the DR group (41%) and the DNR group (36%). This difference rises to a significant level (52% vs. 33%; p=0.0008) by the ninth week of treatment. No such difference appears when the PR group (43%) is compared with the PNR group (41%) after 9 weeks of treatment (p=0.8).

CD8+T cells: As can be seen in FIG. 3C, the percentage of total T cells that were CD8+ trended upward during treatment, with the DNR group tending to have a higher percentage of CD8+ T cells than the DR group. When the ratio of the percentage of CD4+ T cells to the percentage of CD8+ T cells was plotted (see FIG. 3D) over time during the course of treatment, a difference between the DR and DNR groups was observed.

### DISCUSSION

The present invention shows that the induction of oral immune-regulation via oral administration of the study drug, Alequel™, an autologous protein-containing extract of colon mucosal tissue, is a safe and effective treatment for subjects with moderate to severe Crohn's Disease (with a CDAI score between 220 and 400). During the treatment period, 7 out of 12 subjects (58%) in the drug treatment group achieved complete clinical remission (CDAI≦150) for two consecutive time points (a period of 6 weeks), compared to 4 out of 14 of the placebo group (29%). Among those drug-treated subjects who achieved remission, the effect was judged as stable in 3 out of 7 subjects as measured by at least two CDAI scores, indicating remission in the 12 week follow-up period. Clinical response was defined as a decrease of the CDAI score to less than or equal to 100 points from the baseline in two consecutive visits during the study period. Using this standard, 67% of the drug-treated individuals (8 out of 12) achieved clinical response, compared to 6 out of 14 (43%) of the placebo group.

The data of the present invention showed the appearance of an inflection point between 6 and 9 weeks of treatment in the DR population when the CDAI is plotted against time. Such an inflection point is not seen in the DNR treatment group or in either of the placebo groups. In particular, 4 out of 14 PR subjects did not demonstrate such an inflection point. A similar inflection point was observed in a previous Phase I open label trial when the CDAI of the drug responsive group was plotted. Here, the inflection point occurred between 6 and 8 weeks. This is best interpreted as indicating that the onset of efficacy occurs only after this time has elapsed.

As a supplementary measure of safety and efficacy, the data from the Inflammatory Bowel Disease Questionnaire (IBDQ), an evaluation of health perception and function, completed by each subject before and at the end of treatment, was evaluated. The IBDQ score improved an average of 43% in subjects who received the study drug, 61% in the DR cohort, 12% in those who received the placebo and 2% in the PR cohort. When the extent of improvement in quality of life of the DR cohort was compared with those of the PR cohort (2%), a significant difference was observed (61% vs. 2%; p=0.008).

Oral tolerance is an active response towards orally administered immunogenic material. It has been shown in animal model systems that drugs or conditions that interfere with the functioning of the immune system can block the onset of this tolerance. Although the exact mechanism of oral tolerance is not fully understood, it involves the presentation of an epitope to one or more subsets of cells in the gut-associated lymphoid tissue (Strober, S. & Mowat, A. M., Immunol. Today 4, 173-81 (1998)), and a subsequent transport of a second signal that is the result of this epitope-cell interaction into the liver. This process leads to a modulation of the systemic immune response towards the ingested epitope. This mode of therapy has been examined in a number of animal models of immune mediated disorders, including multiple sclerosis, uveitis, rheumatoid arthritis and steatohepatitis (Miller A., Lider, O., Roberts, A. B., Sporn M. & Weiner, H. L., Proc. Natl. Acad. Sci. (USA) 89, 421-425 (1992); Von Herrath, M. G., Dyrberg, T. & Oldstone, M. B. A., J Clin Invest 98, 1324-1331 (1996); Weiner, H. L., Ann Rev Med 48, 341-51 (1997)). The finding in both animal models and in human clinical trials was that the oral administration of antigens may be associated with alteration of the immune balance (manifested in part by an increased immune response) has led to the preferential use of the term “oral immune regulation” by some mucosal immunologists (Ilan, Y., Microbes and Infection 4, 1317-1326 (2002)).

It has been previously shown that the oral administration of hepatitis B envelope proteins in patients with chronic hepatitis B altered the HBV-directed immune response towards a Th1 type profile, by a combination of decreased HBV-specific IL10 producing T cell clones and increased HBV-specific IFNγ-producing T cell clones (Safadi, R. et al., Am J of Gastroenterology 98, 2505-2515 (2003)). In this HBV study, oral immune regulation may have enhanced the effect of either a beneficial subset of T cells towards the ingested antigens, or a regulatory subtype of T lymphocytes that reintroduced the required immunological balance. There may be the simultaneous down regulation of one subset of T-cells and the augmentation of another occurring. Similar approaches were recently described for Schistosoma mansoni infection, in which a dysregulated immune response underlies the disease (McSorley, S. J. & Garside, P., Immunol Today 20, 555-560 (1999); Margalit, M. & Ilan, Y., (AIDS vaccines and related topics) (ed. Bourinbaiar, A. S.) 63-77 (Chachoengsao, Thailand, 2004)).

The oral administration of proteins has been attempted as a therapeutic modality in several human diseases (Husby S., Mestecky, J., Moldoveanu, Z., Holland, S. & Elson, C. O., J Immunolo 152, 4663-4668 (1994)). In general, the results of human trials are less impressive than the results in animal studies, although success has been reported in several models, including uveitis (Safadi R. et al., Am J of Gastroenterology 98, 2505-2515 (2003)). Various factors were proposed as explanations for the variable response observed in humans as compared to animals. These include mode, frequency and dose of administration, specificity and immunogenicity of the ingested antigen, the use of adjuvants, the immuno-genetic background of the patient and inter-individual variability (Wildner, G. & Diedrichs-Mohring, M., Autoimmun Rev 3, 383-387 (2004)).

In a recent study, the induction of oral tolerance towards KLH was examined in normal individuals as well as in those with ulcerative colitis and Crohn's disease (Wu, H. Y. & Weiner, H. L., Immunol Res 28, 265-284 (2003)). The administration of KLH prevented a subsequent T cell proliferative response in the normal individuals who were immunized with KLH. In contrast, in those with ulcerative colitis, and to a greater extent in those with Crohn's disease, the prior oral administration of KLH led to an augmentation rather than a suppression of the T cell proliferative response. Based on these results, it was concluded that oral tolerance cannot be achieved in patients with inflammatory bowel disease using their protocol due to a fundamental defect in the gut associated lymphoid tissue. This conclusion is not necessarily in conflict with the data reported in this work and in the reports of the Phase I trial with Alequel™ since the interval between the administration of the tolerizing agent and the challenge by the KLH was only two weeks. Furthermore, as no in vivo parameters were measured in the KLH study, it remains unclear whether oral administration of disease-associated antigens would induce a clinical effect in patients with Crohn's disease.

In a preferred embodiment of the present invention, differences and changes of several immunological parameters assayed in the course of the cycle of treatment resulted in a significant difference in the immune profile of subjects who respond to treatment when compared both with those subjects who did not respond to treatment as well as those subjects who received placebo. The parameters reported in this results section include the C-reactive protein (CRP), the IFNγ ELISPOT assay, and the levels of NKT, CD4+ and CD8+ cells. Specifically, the level of CRP in the DR group was 1.5 mg/L at the initiation of treatment, while in those treated subjects who did not respond the average CRP level at the initiation of treatment was 6.1 mg/L. This difference between the DR group and the DNR group had a p value of 0.02. Parenthetically, both the PR and the PNR subjects had average CRP scores at the initiation of treatment of 3 mg/L. Secondly, in the DR group of subjects, before treatment, the proportion of T cells that were NKT cells was 2.3%, significantly lower than that of the DNR group (14.8%; p=0.0051). The same difference was not observed when the proportion of NKT cells in the two placebo treated groups was evaluated. For the PR group, the percentage of NKT cells in the T cell population before treatment was initiated was 5%, while for the PNR group the initial percentage was 5.3%. In addition, the percentage of CD4+ cells in the T cell population was higher in DR subjects, compared with DNR subjects (41% vs. 36%), while the percentage of CD8+ cells was lower (18% vs. 25%). Again, this difference was not reflected in the difference between the PR and the PNR group (43% vs. 41%) for CD4+ cells and for the percentage of CD8+ cells (22% vs. 21%). It follows that the ratio of CD4+ to CD8+ cells in the T cell population was higher in the drug responsive cohort (2.2) than in the drug non-responsive cohort (1.3).

This data shows that there is a distinct difference in the immune profile at the beginning of the study in subjects Who were able to respond to the study drug compared with those who did not respond. These findings may have important implications for the ability to predict a response to the study drug.

Certain parameters tracked in the course of this treatment cycle are candidates for potential surrogate markers for clinical response. For example, we found that the IFNγ ELISPOT assay result was correlated with the clinical effect of the study drug in that a decrease in the number of subject-specific antigen induced IFNγ producing SFCs was noted only in the DR group of subjects, i.e., those who received the study drug and achieved clinical remission. Significant alterations in peripheral blood T cell subpopulations over time again correlated with the DR group of subjects. Most notably, a marked increase in the peripheral blood NKT cell number only in this DR group of subjects was found. (2.5% at week 0 to 12.5% at week 9).

Certain parameters tracked in the course of a treatment cycle provided insights into the cellular events that may be taking place at the onset of a therapeutic effect. The temporary rise in the NKT cell number in DR subjects, which decreased towards week 15, may reflect a correlation between the number of this subset of cells and the induction of a clinical response. A significant increase in the number of peripheral CD4 T cells in the drug-treated clinical responders was also seen, along with a significant decrease in the number of CD8 T cells. Although some studies have shown that the induction of tolerance is associated with the transfer of CD8 cells (Kraus, T. A., Tpy, L., Chan, L., Childs, J. & Mayer, L., Gastroenterology 126, 1771-1778 (2004)), other data have implicated the importance of subsets of CD4 cells in this process (Thomas, H. C. & Parrot, M. V., Immunology 27, 631-639 (1974)).

A relatively high placebo effect (29% remission, 43% clinical response) was observed in this study. A recent meta-analysis has identified study duration, number of study visits and disease severity at entry as factors contributing to a high placebo rate (Groux, H. et al., Nature 389, 737 (1997)). In the present study, the relatively long study duration and many clinic visits may have contributed to the placebo effect. Also, all subjects underwent a multiple biopsy colonoscopy session which may in itself have contributed to the placebo effect.

A preferred embodiment of the present invention provides a method for predicting a response to a therapeutic composition for an immune-mediated disease or disorder in a mammalian subject comprising the use of at least one marker or one immune profile marker. The level of these markers may be compared to the level of the marker in a mammalian subject without the disease. These methods may have a positive response, or a negative response. The markers may comprise at least one protein level, T cell level, cytokine level, or any combination thereof. The markers may also comprise C-reactive protein levels, NKT cell levels, CD4+ T cell levels, CD8+ T cell levels, IFNγ-producing cells, or any combination thereof. The C-reactive protein levels may be lower in mammalian subjects with the immune-mediated disease or disorder that respond to drug treatment, or they may be lower in mammalian subjects with said immune-mediated disease or disorder who respond favorably to drug treatment. The NKT cell levels may initially be lower in mammalian subjects with said immune-mediated disease or disorder that respond to drug treatment, or they may be initially lower in mammalian subjects with said immune-mediated disease or disorder who respond favorably to drug treatment. The CD4+ T cell levels may be initially higher in mammalian subjects with said immune-mediated disease or disorder that respond to drug treatment, or they may be initially higher in mammalian subjects with said immune-mediated disease or disorder who respond favorably to drug treatment. The CD8+ T cell levels may be initially higher in mammalian subjects with said immune-mediated disease or disorder that respond to drug treatment, or they may be initially higher in mammalian subjects with said immune-mediated disease or disorder who respond favorably to drug treatment. The number of IFNγ producing cells may be initially higher or the same in mammalian subjects with said immune-mediated disease or disorder that respond to drug treatment, or may be initially higher or the same in mammalian subjects with said immune-mediated disease or disorder who respond favorably to drug treatment.

Another preferred embodiment of the present invention describes a method for predicting a favorable response to a therapeutic composition for an immune-mediated disease or disorder in a mammalian subject comprising the use of markers in said subject comprising low C-reactive protein levels as compared to a normal mammalian subject, low NKT cell levels as compared to a normal mammalian subject, high CD4+ cell levels as compared to a normal mammalian subject, high CD8+ T cell levels as compared to a normal mammalian subject, high IFNγ cell levels as compared to a normal mammalian subject, or any combination thereof.

The present invention also provides a method for creating an immune profile using markers to predict a positive response to a drug, to autologous colon-extracted antigens, or to autologous protein-containing extract of colon mucosal tissue for treating an immune-mediated disorder or disease.

An additional preferred embodiment describes a method for creating an immune profile for patients with Crohn's disease using markers to predict a positive response to Alequel™. The positive responses described may consist of clinical remission, clinical response, improved quality of life or a CDAI score of ≦150.

TABLE 1
Demographic and clinical characteristics of subjects at
initiation of treatment

| | Study drug (n = 12) | Placebo (n = 14) |
|---|---|---|
| Sex M:F | 3:9 | 7:7 |
| Age | 30.6 ± 6.7 (20-47) | 33.0 ± 12.5 (18-56) |
| Duration of disease | 8.7 years | 8.6 years |
| Location of disease | | |
| Small bowel | 4 | 5 |
| Colon | 2 | 1 |
| Both | 6 | 8 |
| Steroid treatment | N = 4 | N = 3 |
| Baseline CDAI | 310 ± 54.4 | 292 ± 54.5 |
| Baseline IBDQ | 116 ± 13.7 | 134 ± 22.7 |

TABLE 2
Summary of response rate:

| | | | Quality of life |
|---|---|---|---|
| | Clinical | Clinical | Average % |
| | response | remission | improvement |
| Drug treated group | 67% | 58% | 43% |
| Placebo treated | 43% | 29% | 12% |
| group | | | |

1. A method for predicting a response to a therapeutic composition for an immune-mediated disease or disorder in a mammalian subject comprising the use of at least one marker.

2. A method for predicting a response to a drug in a mammalian subject with an immune mediated disease or disorder comprising the use of at least one immune profile marker.

3. The method of claim 1 or 2 wherein the level of said marker in said mammalian subject with said immune-mediated disease is compared to the level of said marker in a mammalian subject without said disease.

4. The method of claim 3 wherein said response is positive.

5. The method of claim 3 wherein said response is negative.

6. The method of claim 1, 2, 3, 4 or 5 wherein said markers comprise at least one protein level, T cell level, cytokine level, or any combination thereof.

7. The method of claim 1, 2, 3, 4 or 5 wherein said markers comprise C-reactive protein levels, NKT cell levels, CD4+ T cell levels, CD8+ T cell levels, IFNγ-producing cells, or any combination thereof.

8. The method of claim 1, 2, 3, or 4 wherein said C-reactive protein levels are lower in mammalian subjects with said immune-mediated disease or disorder that respond to drug treatment.

9. The method of claim 1, 2 or 3 wherein said C-reactive protein levels are lower in mammalian subjects with said immune-mediated disease or disorder who respond favorably to drug treatment.

10. The method of claim 1, 2, 3 or 4 wherein said NKT cell levels are initially lower in mammalian subjects with said immune-mediated disease or disorder that respond to drug treatment.

11. The method of claim 1, 2 or 3 wherein said NKT cell levels are initially lower in mammalian subjects with said immune-mediated disease or disorders who respond favorably to drug treatment.

12. The method of claim 1, 2, 3 or 4 wherein said CD4+ T cell levels are initially higher in mammalian subjects with said immune-mediated disease or disorder that respond to drug treatment.

13. The method of claim 1, 2 or 3 wherein said CD4+ T cell levels are initially higher in mammalian subjects with said immune-mediated disease or disorder who respond favorably to drug treatment.

14. The method of claim 1, 2, 3 or 4 wherein said CD8+ T cell levels are initially higher in mammalian subjects with said immune-mediated disease or disorder that respond to drug treatment.

15. The method of claim 1, 2 or 3 wherein said CD8+ T cell levels are initially higher in mammalian subjects with said immune-mediated disease or disorder who respond favorably to drug treatment.

16. The method of claim 1, 2, 3 or 4 wherein said number of IFNγ producing cells are initially higher or the same in mammalian subjects with said immune-mediated disease or disorder that respond to drug treatment.

17. The method of claim 1, 2 or 3 wherein said number of IFNγ producing cells are initially higher or the same in mammalian subjects with said immune-mediated disease or disorder who respond favorably to drug treatment.

18. A method for predicting a favorable response to a therapeutic composition for an immune-mediated disease or disorder in a mammalian subject comprising the use of markers in said subject comprising:
a) low C-reactive protein levels as compared to a normal mammalian subject;
b) low NKT cell levels as compared to a normal mammalian subject;
c) high CD4+ cell levels as compared to a normal mammalian subject;
d) high CD8+ T cell levels as compared to a normal mammalian subject;
e) high IFNγ cell levels as compared to a normal mammalian subject; or
f) any combination thereof.

19. A method for creating an immune profile using markers to predict a positive response to a drug for treating an immune-mediated disorder or disease.

20. A method for creating an immune profile using markers to predict a positive response to autologous colon-extracted antigens for treating an immune-mediated disorder or disease.

21. A method for creating an immune profile using markers to predict a positive response to autologous protein-containing extract of colon mucosal tissue for treating an immune-mediated disorder or disease.

22. A method for creating an immune profile for patients with Crohn's disease using markers to predict a positive response to Alequel.

23. The method of claims 4, 18-22 wherein said positive response comprises clinical remission, clinical response, improved quality of life or a CDAI score of ≦150.
